(19) United States
(12) Patent Application Publication
(10) Pub. No.: US 2017/0119559 A1
(43) Pub. Date: May 4, 2017

(54) A Ureteral Stent

(71) Applicant: Coloplast A/S, Humlebaek (DK)

(72) Inventors: Sebastien Seguy, Gourdon (FR); Thomas Beilvert, Saint Martial de Nabirat (FR); Jacques Millet, Sarlet la Canéda (FR); Chaabane Bougherara, Frederiksberg C (DK); Olivier Chouarche, Vitrac (FR)

(21) Appl. No.: 15/302,021

(22) PCT Filed: Apr. 10, 2015
(86) PCT No.: PCT/DK2015/050088

§ 371 Date: Oct. 5, 2016

(30) Foreign Application Priority Data
Apr. 11, 2014 (EP) 14290106.5
Jul. 23, 2014 (EP) 14178203.7

(51) Int. Cl. A61F 2/94; A61M 27/00

(52) U.S. Cl. CPC A61F 2/94; A61M 27/008; A61F 2002/048

(57) ABSTRACT

The present invention relates to a ureteral stent (10) having a body (11) and a tail (12). The body (11) of the stent has a renal section (13) intended to be placed in a kidney (R) of a patient, a ureteral section (14) intended to be placed in at least part of a ureter of said patient, and a proximal section (15) arranged at a proximal end of the body of the stent. The tail (12) has at least one thread (16) intended to end in the bladder (V) of said patient. This stent is characterized in that said proximal section (15) has a flexibility greater than the flexibility of the ureteral a section (14) of the stent. The tail (12) is integral with said ureteral section and/or said proximal section (15) and extends beyond the proximal section (15).

### BACKGROUND

The present invention relates to a ureteral stent intended to maintain the flow of urine between the kidneys and the bladder of a patient.

In a person, urine secreted by the kidneys passes through the ureters to the bladder and is then evacuated from the body through the urethra during micturition. In a healthy person, the urine is evacuated from the kidney to the bladder in one direction, by means of peristaltic movements of the ureter.

Certain urological disorders or certain diseases may prevent this evacuation in the direction of the bladder. These disorders may in particular be due to the presence of a calculus or tumour or an obstruction of the pyeloureteral junction. In this case, the flow of urine to the bladder may be difficult or may no longer be possible at all. The urine remains in the kidney, which dilates and may cause nephritic colic. To remedy this disorder, a stent can be placed in the ureter in order to re-establish the function of the latter and to permit evacuation of the urine.

### BRIEF SUMMARY

The application relates to a ureteral stent according to claim 1.

### BRIEF DESCRIPTION OF THE DRAWINGS

Embodiments and advantages will be better understood with reference to the detailed description and to the attached figures, in which:

FIG. 1 is a schematic view of one embodiment of a stent placed in a patient;

FIGS. 2 and 3 are front views of embodiments used to fix a tail to a stent;

FIG. 4 is a profile view of part of one embodiment of a ureteral stent;

FIG. 5 is a sectional view of part of the stent, according to one alternative embodiment;

FIG. 6 is a cross-sectional view of the embodiment of FIG. 5;

FIG. 7 is a backside view of part of the stent, according to one embodiment;

FIG. 8 is a cross-sectional view of the embodiment of FIG. 7;

FIGS. 9 to 11 are sectional views of embodiments;

FIGS. 12, 14 and 16 to 18 are schematic perspective views of embodiments;

FIG. 13 is a cross-sectional view of the embodiment of FIG. 12; and

FIG. 15 is a cross-sectional view of the embodiment of FIG. 14.

### DETAILED DESCRIPTION

The ureteral stent according to this application includes a body and a tail, the body having a renal section for placement in a kidney of a patient, a ureteral section for placement in a ureter of the patient, and a proximal section arranged at a proximal end of the body of the stent, the tail having at least one thread configured to end in the bladder of said patient.

The present invention provides a stent which preserves the peristaltic movements of the ureter, thereby preventing reflux of urine in the direction of the kidneys. The stent is configured to avoid irritation of the bladder and irritation caused by friction in the ureter. The stent is provided to be better tolerated by patients. The tail has at least one thread permitting easy and optimal withdrawal of the stent. In one embodiment, the tail is additionally fixed to the stent to further optimize its functionality including guiding of urine without causing irritation while preventing reflux and permitting withdrawal of the stent.

The ureteral stent includes a proximal section that has a first flexibility greater than a second flexibility of the ureteral section of the stent. The tail is integral with the ureteral section, and extends beyond the proximal section.

The ureteral stent is useful for placement in a ureter of a patient suffering from a urological disorder or disease, such as a calculus, a tumour, or an obstruction of the pyeloureteral junction in particular. The stent extends as far as the kidney and has a curved renal section, the curved shape allowing the stent to be held in place in the kidney. The stent has a ureteral section configured to be inserted in the ureter of the patient. The ureteral section extends beyond the area in which the patient suffers from a disease or disorder, and it ensures a flow path in the defective part of the ureter. One end of the stent is provided with a tail which comprises at least one thread configured to end in or near the bladder. The thread is suitable for permitting evacuation of the urine and has a sufficiently small diameter to be virtually physically imperceptible to the patient.

The greater first flexibility of the proximal section over the second flexibility of the ureteral section greatly reduces or completely eliminates any discomfort in the patient caused by the presence of the stent. Moreover, the thread of the stent permits easier withdrawal of the stent for removal.

In the following disclosure, the features of the various exemplary embodiments are suitable to be combined with each other unless specifically noted otherwise.

In the following disclosure, end means an end-most location and end portion means that segment adjacent to and near the end of an object.

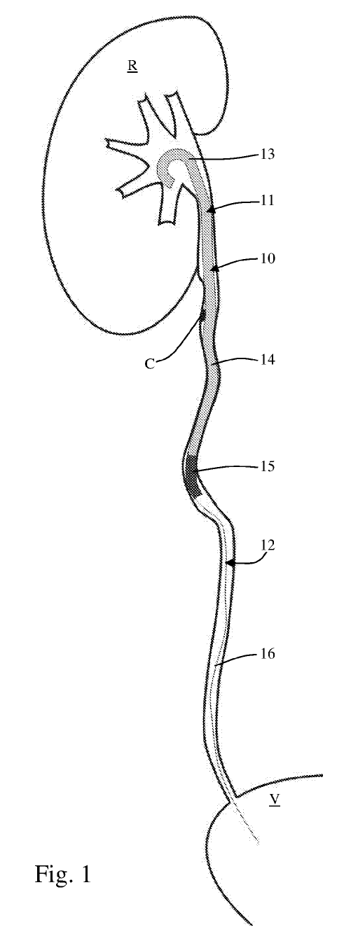

FIG. 1 is a schematic view of one embodiment of a ureteral stent 10, placed in a patient presenting a calculus C. The ureteral stent 10 includes a body 11 and a tail 12. The body has a renal section 13 configured to be placed in a kidney R of a patient. The renal section 13 includes a curved end configured to prevent the stent from shifting in the ureter once in place. This part of the stent is physically imperceptible or virtually imperceptible to the patient and, as a result, does not cause any discomfort or pain.

In one embodiment, the stent has a ureteral section 14 for placement in a ureter of the patient. Embodiments include stents manufactured with varying lengths of at least the ureteral section such as to accommodate variations in patient physiology. As has been indicated above, a ureteral stent of this kind is suitable for placement in a patient in cases of disease or other obstruction in the area of the ureter. The obstruction may be due to the presence of a calculus C, as is illustrated by FIG. 1, a tumour or a constriction in particular. The length of the ureteral section 14 of the stent should be sufficient to ensure that, after placement of the stent, the body 11 thereof extends beyond the site of the calculus or the tumour in the direction of the bladder.

In one embodiment, the body 11 of the stent additionally has a proximal section 15 which is integral with an end of the ureteral section 14, at the end away from the renal section 13. The proximal section 15 is situated in continuation of the ureteral section 14.

In one embodiment, the tail 12 of the stent includes at least one thread or suture 16, which is configured to extend from the proximal section 15 in the direction of the bladder V, when the stent is placed in a patient.

In one embodiment, the tail 12 is configured to end in the bladder and extends a few centimetres into the bladder when the stent is placed in a patient. In one embodiment, the tail extends 5 to 6 centimetres into the bladder.

In embodiments wherein the tail is formed by two or more threads, the threads may suitably be free and independent of one another, or alternatively fixed to one another, for example by means of a knot. The knot is suitably located near the proximal section 15, in which case it will be positioned in the ureter during the use of the stent. Alternatively, the knot is located near the ends of the threads extending away from the proximal section, in which case it will be positioned in the bladder during the use of the stent. Other ways of connecting the threads to one another are also acceptable, such as, but not limited to braiding.

Providing a thread, even of small diameter, has several effects. One of the effects is to permit the flow of urine from the kidney R to the bladder V without permitting flow in the opposite direction from the bladder to the kidney. This reduces or eliminates problems concerning, in particular, the sensation of having to pass urine.

Another effect of the thread is that it causes a dilation of the ureter, even if the thread has a small diameter in relation to the ureter. Such dilation permits easier and therefore less painful evacuation of the calculi.

In one embodiment, the ureteral section 14 and the proximal section 15 are tubular and cylindrical, that is to say they have a substantially annular cross section, with a longitudinal channel 17 defined by a lateral wall 18. In one embodiment, the lateral wall has, at least in some areas, through-openings (not shown) which allow the urine to flow from outside the stent to the inside of the longitudinal channel, and vice versa. In one embodiment, the longitudinal channel 17 opens out at the end of the proximal section 15, near the tail 12. In one embodiment, the renal section 13 also has a channel and openings passing through the lateral wall 19. The channel serves, during the placement of the stent, for introduction of a guide and, during the use of the stent, for evacuation of urine.

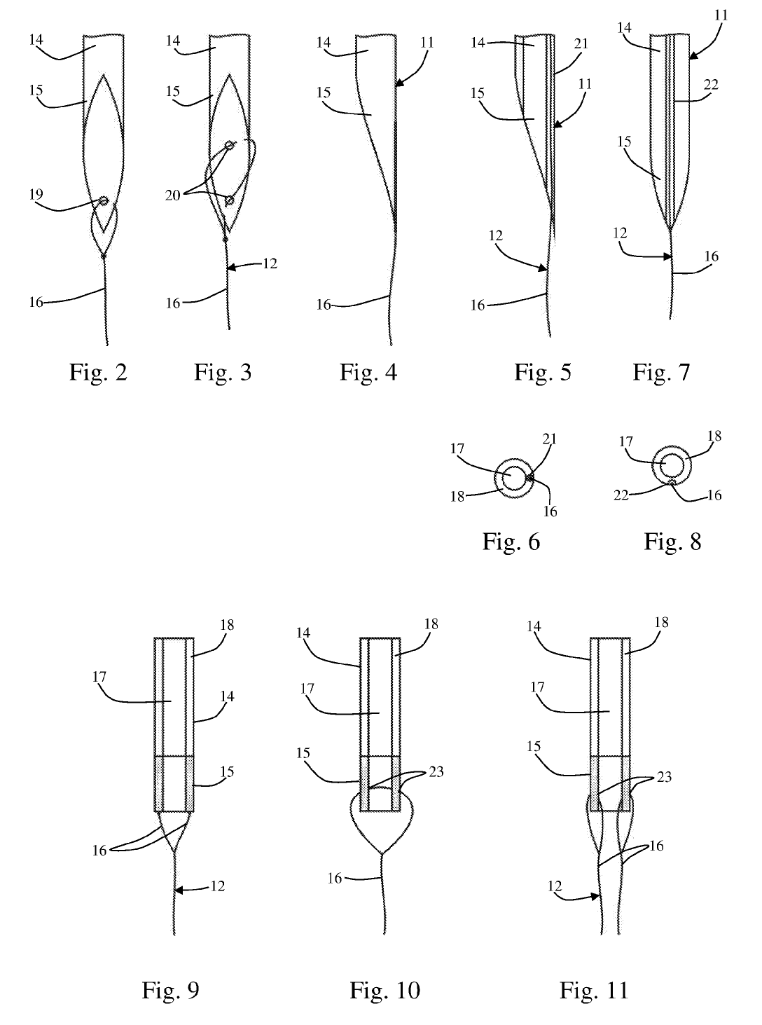

In embodiments illustrated in FIGS. 2 to 8, the proximal section 15 has a bevelled shape, this shape providing flexibility to the proximal section. In the embodiments illustrated in FIGS. 2 to 6, the tail 12 of the stent includes a thread 16 which is fixed in the proximal section 15 of the stent.

In this application, flexibility is defined here as the resistance to elastic deformation of a body. The more a body is flexible, the less the force that has to be applied to it to obtain a given deflection. In the present specification, stiffness or rigidity is considered the inverse of flexibility.

The flexibility can be measured in the following way: a specimen for which flexibility is being determined is placed on two punctiform supports that are separated by a distance L. A downward force P is applied in the centre of the specimen. The deformation U_{y} of the specimen is measured by measuring the movement of the centre of the specimen under the effect of the force P. This deformation, as a function of the force, allows a curve to be established. The coefficient of flexibility is defined as being the slope of the tangent to this curve, at the origin.

From the mathematical point of view, the coefficient of flexibility is defined by

$$K = \frac{{L}^{3}}{48} \cdot \frac{P}{\vert {U}_{y} \vert}$$

In one embodiment, the coefficient of flexibility of the proximal section of the stent is less than or equal to 200 N mm².

The more flexible the stent, the greater the flexibility and the lower the coefficient of flexibility.

Flexibility of the proximal section is suitably obtained by use of flexible materials and/or by providing the proximal section with a shape that gives it flexibility.

In one embodiment, the proximal section of the stent is configured to allow the body of the stent to match the shape of a ureter, in particular in non-rectilinear parts of the ureter, during movements of the patient, especially movements caused by respiration. The stent and the ureter are configured to allow relative shifting between them without irritating or injuring the ureter. For this purpose, the proximal section of the stent is sufficiently flexible to be able to follow the ureter without inflicting injuries on the latter.

A suitable material for producing the proximal section can be chosen among different types of polymers such as polyurethane, copolymers such as the polyether block amide “PEBA”, polyvinyl chloride, polyamides or silicone in particular, or more generally from among the materials mentioned below for the ureteral section.

In one embodiment, the ureteral section of the stent has a flexibility allowing it to adapt to the sinuosities of the ureter. Materials suitable for producing the stent include polymers such as polyurethane, copolymers such as polyether block amide known by the name PEBA, polyamides, silicone, polyolefins sold under the names Infuse, Vistamaxx™, Queo™ or Notio™; polyamides, PVC, thermoplastic polyurethanes, aromatic polyethers, aromatic and aliphatic polyesters having a Shore hardness of generally between 25 and 95; compounds based on thermoplastic elastomers, vulcanized thermoplastic elastomers, mixtures and alloys based on thermoplastic polyurethane, polymers and copolymers sold under the names Thermoflex™, Hytril™, Arnitel™, EVA, and thermoplastic elastomers known by their acronyms SIS, SEBS, SEPS, SEEPS, SBS, SIBS or SIBSTAR.

In embodiments, an external diameter of the ureteral stent is between 1.5 mm and 4 mm.

Suitable materials for the thread (or suture) include: polyethylene, polyamide, polyester, silk, steel, resorbable material (such as polyglactin acid), high-density polypropylene, meta-aramid and para-aramid, such as Kevlar™ or Nomex™.

In embodiments, the thread is configured to have a diameter ranging between 0.16 mm and 1.3 mm. In one embodiment, the diameter is substantially equal to 0.2 mm.

In one embodiment illustrated in FIG. 2, an end of the proximal section 15 of the stent has a through-opening 19. A thread 16 passes through the through-opening 19 and forms a loop, which is fixed to another part of the thread, for example by a knot.

In one embodiment, the through-opening 19 is located relatively close to a free end of the proximal section 15, the free end defined as the end of the proximal section 15 away from the ureteral section 14.

In one embodiment illustrated in FIG. 3, the proximal section 15 has two openings 20 arranged at different distances from the free end of the proximal section 15. The thread 16 passes through the two openings 20, and one of the ends of the thread is fixed to another part of the thread. The fixation is suitably obtained by a knot, by adhesive bonding or by a combination of these.

One advantage of the embodiment of FIG. 3 is that it avoids rolling-up of the stent during withdrawal and unintentional tearing of the proximal section when the thread is pulled. It should be noted that the openings 20 are suitably placed with one in the proximal section 15 and the other in the ureteral section 14.

In one embodiment illustrated in FIG. 4, the thread 16 is arranged in a continuation of the tapered end of the proximal section 15. The thread is suitably provided separately from the body 11 of the stent and configured for subsequent attachment thereto. The tail is suitably fixed by adhesive bonding or by welding. In one embodiment, one or more threads are fixed by ultrasonic welding. In this context, it is possible to use an ultrasound-generating cone in the body of the stent and in this way weld the thread.

In one embodiment, the thread 16 is suitably produced at the same time as the body 11 of the stent, for example by co-extrusion or drawing of the material forming the body. Thereby, there is practically no transition between the body 11 of the stent and, in particular, between the proximal section 15 and the tail 12 of the stent.

In embodiments illustrated in FIGS. 5 to 8, a thread 16 opens out at the tapered end of the proximal section 15 with a gentle, or smooth, transition between the body 11 of the stent and the tail 12.

In the embodiments of FIGS. 5 and 6, the proximal section 15 includes a channel 21 in which the thread 16 is arranged. The thread is suitably held in the channel 21 by adhesive bonding or welding. In embodiments, the channel 21 suitably extends into the ureteral section 14.

In the embodiments illustrated in FIGS. 7 and 8, the proximal section 15 includes a groove 22 in which the thread 16 of the tail is arranged. The thread is suitably fixed to the body 11 by welding or adhesive bonding. In embodiments, the groove 22 suitably extends into the ureteral section 14.

In embodiments illustrated in FIGS. 9 to 18, a first flexibility of the proximal section 15 is not provided by the bevelled shape but instead by the material or cut-outs made in this proximal section.

In the embodiments illustrated in FIGS. 9 to 11, the first flexibility of the proximal section 15 is provided by use of a flexible material. In the embodiment illustrated in FIG. 9, the tail 12 has two strands of thread 16, which are fixed to a free end of the proximal section. The fixation is suitably obtained by adhesive bonding or welding. The two strands join to form one thread. In other embodiments, the threads do not join so that tail 12 includes more than one thread 16. In one embodiment, more than two threads are used and fixed to the proximal section 15.

In one embodiment illustrated in FIG. 10, the proximal section 15 has two through-openings 23 arranged substantially at the same distance from a free end of the proximal section. The thread 16 forming the tail passes through these through-openings 23 and forms a loop. The loop can be closed by a knot and/or by adhesive bonding. It is also possible to provide more than two through-openings.

In one embodiment illustrated in FIG. 11 , the tail 12 includes two threads 16 and each thread 16 of passes through a single through-opening 23. It is possible to provide a tail formed by a single thread passing through a single opening or, by contrast, more than two threads.

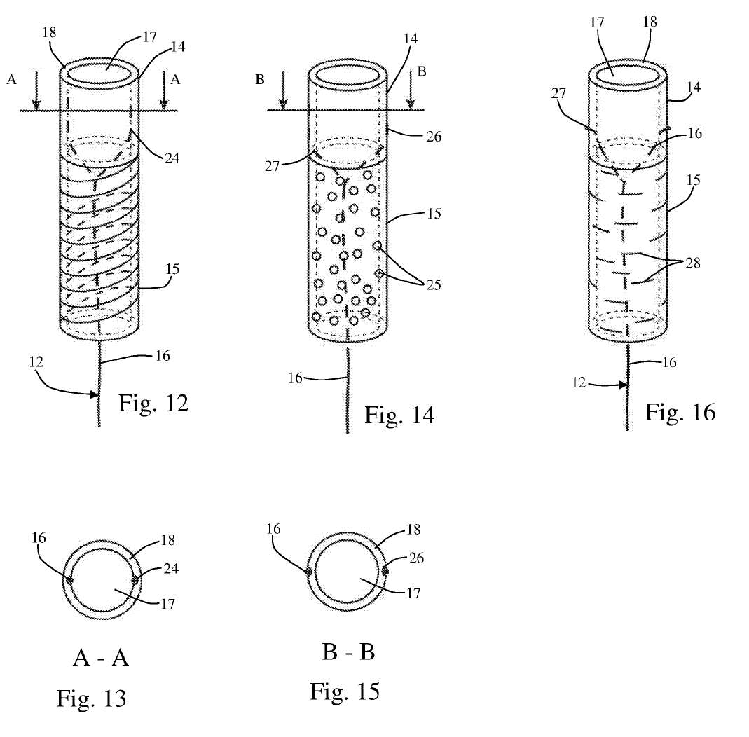

In embodiments illustrated in FIGS. 12 to 18, a first flexibility of the proximal section 15 is provided by removing material from the proximal section, as is explained in detail below.

In one embodiment illustrated in FIGS. 12 and 13, the proximal section 15 is formed by a spiral. This spiral shape provides the proximal section of the stent with high flexibility. In this embodiment, the tail 12 is not fixed to the proximal section 15, but to the ureteral section 14. At least part of the ureteral section 14 includes one or more internal grooves 24. A thread 16 of the tail is suitably arranged in each groove and is fixed therein. The fixation is suitably obtained by adhesive bonding or welding.

In the embodiment illustrated in FIGS. 14 and 15, the proximal section 15 includes a plurality of through-openings 25. At least one effect of the openings is that they make the proximal section more flexible. The ureteral section 14 has, at least in a part near the proximal section 15, two or more external longitudinal grooves 26 and two through-openings 27 connected to the external grooves 26. At least one thread 16 forming the tail passes through the openings 27 and is placed in the external grooves 26. The tail continues beyond the proximal section 15. The threads arranged in the external grooves 26 are fixed to the body of the stent by knots, adhesive bonding and/or welding. Other ways of fixing the threads to the body are also acceptable.

During normal use of the stent of this embodiment, urine is able to flow freely along the tail 12 without the tail interfering with the proximal section 15. During withdrawal of the stent, the pull applied to the tail acts on the ureteral section 14, which makes it possible to exert a certain amount of force without deforming the proximal section 15. Moreover, the proximal section retains its flexibility during withdrawal of the stent.

In one embodiment illustrated in FIG. 16, the proximal section 15 includes transverse slits 28 provided to make the proximal section flexible. The ureteral section 14 includes at least two through-openings 27 arranged near the proximal section 15. A thread 16 forming the tail 12 passes through these openings 27 and is fixed to the body of the stent. The fixation is suitably obtained by a knot, welding and/or adhesive bonding.

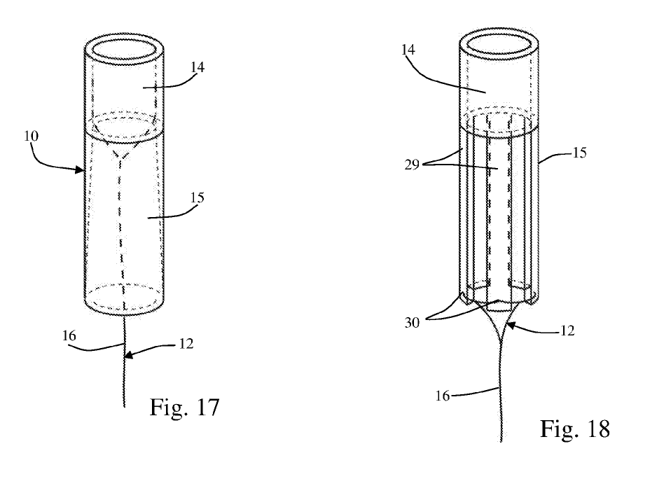

FIG. 17 shows one embodiment of a stent 10 in which the proximal section 15 has a wall thickness that decreases towards a free end of the proximal section. The reduction in wall thickness increases the flexibility of the proximal section.

The tail 12 is fixed to the ureteral section 14. The fixation can be obtained by adhesive bonding, welding, coextrusion or by a knot.

In embodiments, the different shapes and different ways of fixing the tail 12 to the body 11 of the stent 10 may be combined. The external groove 26 may suitably be applied to any of the embodiments illustrated in FIGS. 12 to 17. The same applies to the other ways of fixing the tail to the body.

In one embodiment illustrated in FIG. 18, the proximal section 15 is formed by tongues 29 each having a through-opening 30 placed near the free end of the proximal section 15. The tail 12 is provided by thread 16 which passes through the through-openings 30 of the tongues 29 and connects all of these. The tail 12 continues beyond the proximal section 15.

In this embodiment, the tongues 29 are flexible during normal use of the stent. Urine can flow along the tail 12 of the stent without the possibility of a reflux movement.

When the stent of this embodiment is withdrawn, a pull applied to the tail effectively draws the tongues 29 together. This prevents the tongues from rolling up and reduces the external diameter of the stent at the end of the body of the stent to make withdrawal of the stent easier.

A stent has been described which permits evacuation of urine from the patient while at the same time preventing reflux of the urine in the direction towards the kidneys. The stent also dilates the ureter, which permits easier evacuation of calculi. The stent is virtually imperceptible to the patient because the proximal section is flexible. The thread is fine (of small diameter) and flexible, which means that it is practically unnoticed by the patient. Moreover, the tail is fixedly secured to the body of the stent, which allows for easy withdrawal of the stent.

### Embodiments

A. A ureteral stent comprising:
a body having a renal area configured to be placed in a kidney of a patient, a ureteral area connected to the renal area and configured to be placed in a ureter of the patient, and a proximal area connected to the ureteral area and located at a proximal end of the body; and
a tail comprising a thread connected to the stent; wherein
the proximal area is provided with a first flexibility that is greater than a second flexibility of the ureteral area of the stent and in that the tail is rigidly connected to the ureteral area and extends beyond the proximal area.

B. The ureteral stent according to embodiment A, wherein the ureteral area comprises one or more through-openings, and the tail is fixed to the body of the stent via one or more of the through-openings.

C. The ureteral stent according to embodiment A or B, wherein the tail is fixed to the body by a knot.

D. The ureteral stent according to embodiment A or B, wherein the tail is fixed to the body by adhesive bonding.

E. The ureteral stent according to embodiment A or B, wherein the tail is fixed to the body by welding.

F. The ureteral stent according to embodiment B, wherein the tail passes through two or more of the through-openings.

G. The ureteral stent according to embodiment A, wherein the ureteral area includes a groove into which the tail is connected.

H. The ureteral stent according to embodiment G, wherein the groove is arranged outside the body of the stent.

I. The ureteral stent according to embodiment G, wherein the one or more grooves are arranged inside the body of the stent.

J. A ureteral stent comprising:
a body having a renal area configured to be placed in a kidney of a patient, a ureteral area connected to the renal area and configured to be placed in a ureter of the patient, and a proximal area connected to the ureteral area and located at a proximal end of the body; and
a tail comprising a thread connected to the stent; wherein
the proximal area is provided with a first flexibility that is greater than a second flexibility of the ureteral area of the stent and in that the tail is rigidly connected to the proximal area and extends beyond the proximal area.

K. The ureteral stent according to embodiment J, wherein the proximal area comprises one or more through-openings and the tail is fixed to the body via one or more of the through-openings.

L. The ureteral stent according to embodiment J or K, wherein the tail is fixed to the body by a knot.

M. The ureteral stent according to embodiment J or K, wherein the tail is fixed to the body by adhesive bonding.

N. The ureteral stent according to embodiment J or K, wherein the tail is fixed to the body by welding.

O. The ureteral stent according to embodiment J, wherein the proximal area includes a channel into which the tail is introducible.

P. The ureteral stent according to embodiment J, wherein the proximal area includes a groove into which the tail is introducible.

Q. The ureteral stent according to embodiment J, wherein the proximal area comprises several tongues, each having one or more openings sized for the tail of the stent to pass through.

R. Ureteral stent (10) having a body (11) and a tail (12), the body (11) having a renal area (13) intended to be placed in a kidney (R) of a patient, a ureteral area (14) intended to be placed in at least part of a ureter of said patient, and a proximal area (15) arranged at a proximal end of the body of the stent, the tail (12) having at least one thread (16) intended to end in the bladder (V) of said patient, characterized in that said proximal area (15) has a flexibility greater than the flexibility of the ureteral area (14) of the stent, in that the tail (12) is rigidly connected to said ureteral area (14), and in that it extends beyond said proximal area (15).

S. Ureteral stent according to embodiment R, characterized in that the ureteral area (14) has at least one through-opening (27), and in that the tail (12) is fixed to the body (11) of the stent via said at least one through-opening (27).

T. Ureteral stent according to embodiment R or S, characterized in that the tail (12) is fixed by means of a knot.

U. Ureteral stent according to embodiment R or S, characterized in that the tail (12) is fixed by adhesive bonding.

V. Ureteral stent according to embodiment R or S, characterized in that the tail (12) is fixed by welding.

W. Ureteral stent according to embodiment R, characterized in that the ureteral area (14) has at least two through-openings (27), and in that the tail (12) passes through at least two of said at least two through-openings (27).

X. Ureteral stent according to embodiment R, characterized in that the ureteral area (14) has at least one groove (24, 26) into which a part of said tail (12) is introduced.

Y. Ureteral stent according to embodiment X, characterized in that said groove (26) is arranged outside the body (11) of the stent.

Z. Ureteral stent according to embodiment X, characterized in that said groove (24) is arranged inside the body (11) of the stent.

AA. Ureteral stent (10) having a body (11) and a tail (12), the body (11) having a renal area (13) intended to be placed in a kidney (R) of a patient, a ureteral area (14) intended to be placed in at least part of a ureter of said patient, and a proximal area (15) arranged at a proximal end of the body of the stent, the tail (12) having at least one thread (16) intended to end in the bladder (V) of said patient, characterized in that said proximal area (15) has a flexibility greater than the flexibility of the ureteral area (14) of the stent, in that the tail (12) is rigidly connected to said proximal area (15), and in that it extends beyond this proximal area (15).

BB. Ureteral stent according to embodiment AA, characterized in that the proximal area (15) has at least one through-opening (19, 20, 23, 30), and in that the tail (12) is fixed to the body via said at least one through-opening.

CC. Ureteral stent according to embodiment AA or BB, characterized in that the tail (12) is fixed by means of a knot.

DD. Ureteral stent according to embodiment AA or BB, characterized in that the tail (12) is fixed by adhesive bonding.

EE. Ureteral stent according to embodiment AA or BB, characterized in that the tail (12) is fixed by welding.

FF. Ureteral stent according to embodiment AA, characterized in that the proximal area (15) has a channel (21) into which the tail (12) is introduced.

GG. Ureteral stent according to embodiment AA, characterized in that the proximal area (15) has a groove (22) into which the tail (12) is introduced.

HH. Ureteral stent according to embodiment AA, characterized in that the proximal area has several tongues (29), these tongues (29) each having at least one opening (30) through which the tail (12) of the stent (10) passes.

What is claimed is:

1. A ureteral stent comprising:
a body having a renal section configured to be placed in a kidney of a patient, a ureteral section connected to the renal section and configured to be placed in a ureter of the patient, and a proximal section connected to the ureteral section at a proximal end of the body; and
a tail comprising a thread connected to the stent; wherein
the proximal section is provided with a first flexibility that is greater than a second flexibility of the ureteral section of the stent and in that the tail is integral with the ureteral section and extends beyond the proximal section.

2. The ureteral stent according to claim 1, wherein the ureteral section comprises one or more through-openings, and the tail is fixed to the body of the stent via one or more of the through-openings.

3. The ureteral stent according to claim 1 or 2, wherein the tail is fixed to the body by a knot.

4. The ureteral stent according to claim 1 or 2, wherein the tail is fixed to the body by adhesive bonding.

5. The ureteral stent according to claim 1 or 2, wherein the tail is fixed to the body by welding.

6. The ureteral stent according to claim 2, wherein the tail passes through two or more of the through-openings.

7. The ureteral stent according to claim 1, wherein the ureteral section includes a groove into which the tail is connected.

8. The ureteral stent according to claim 7, wherein the groove is arranged outside the body of the stent.

9. The ureteral stent according to claim 7, wherein the one or more grooves are arranged inside the body of the stent.

10. A ureteral stent comprising:
a body having a renal section configured to be placed in a kidney of a patient, a ureteral section connected to the renal section and configured to be placed in a ureter of the patient, and a proximal section connected to the ureteral section and located at a proximal end of the body; and
a tail comprising a thread connected to the stent; wherein
the proximal section is provided with a first flexibility that is greater than a second flexibility of the ureteral section of the stent and in that the tail is integral with the proximal section and extends beyond the proximal section.

11. The ureteral stent according to claim 10, wherein the proximal section comprises one or more through-openings and the tail is fixed to the body via one or more of the through-openings.

12. The ureteral stent according to claim 10 or 11, wherein the tail is fixed to the body by a knot.

13. The ureteral stent according to claim 10 or 11, wherein the tail is fixed to the body by adhesive bonding.

14. The ureteral stent according to claim 10 or 11, wherein the tail is fixed to the body by welding.

15. The ureteral stent according to claim 10, wherein the proximal section includes a channel into which the tail is introducible.

16. The ureteral stent according to claim 10, wherein the proximal section includes a groove into which the tail is introducible.

17. The ureteral stent according to claim 10, wherein the proximal section comprises several tongues, each having one or more openings sized for the tail of the stent to pass through.
